(19) United States
(12) Patent Application Publication
(10) Pub. No.: US 2026/0033884 A1
(43) Pub. Date: Feb. 5, 2026

(54) INTRACARDIAC CATHETER WITH X-RAY EMITTING PROBE

(71) Applicant: BIOSENSE WEBSTER (ISRAEL) LTD., Yokneam (IL)

(72) Inventor: Assaf Govari, Haifa (IL)

(21) Appl. No.: 18/997,168

(22) PCT Filed: Jun. 13, 2023
(86) PCT No.: PCT/IB2023/056077

§ 371 Date: Jan. 20, 2025

Related U.S. Application Data
(60) Provisional application No. 63/390,324, filed on Jul. 19, 2022.

(51) Int. Cl. A61B 18/12; A61B 18/00; A61B 18/14; H05G 2/00

(52) U.S. Cl. CPC A61B 18/1206; A61B 18/1492; H05G 2/001; A61B 2018/0022; A61B 2018/00375; A61B 2018/00577; A61B 2018/00642; A61B 2018/00839; A61B 2018/122

(57) ABSTRACT

A system (20) for tissue ablation includes a probe (22), an expandable capsule (50), a voltage discharge device (210), and a generator (49). The probe is configured to be inserted into a cavity of an organ of a patient. The expandable capsule is fitted at a distal end of the probe and configured to be expanded within the cavity and to be filled with a gas (250). The voltage discharge device is fitted inside the expandable capsule and is configured to create plasma by electrical excitation of the gas that fills the expandable capsule, the plasma emitting X-rays (270), so as to ablate tissue in the cavity using the X-rays. The generator is wired to the voltage discharge device to apply electrical signals that electrically excite the plasma.

### CROSS-REFERENCE TO RELATED APPLICATION

This application claims the benefit of U.S. Provisional Patent Application 63/390,324, filed Jul. 19, 2022, which is incorporated herein by reference.

### FIELD OF THE DISCLOSURE

The present disclosure relates generally to medical devices, and particularly to methods and systems for ablating tissue using intrabody probe producing X-rays.

### BACKGROUND OF THE DISCLOSURE

Intrabody probes that emit X-rays have been proposed in the patent literature. Typically, such probes comprise a miniature X-ray tube. For example, U.S. Pat. No. 6,148,061 describes a miniature x-ray unit that includes a first electrical node, a second electrical node and an insulating material not extending into a vacuum gap between the first and second electrical nodes. Recessing the insulating material from the vacuum gap decreases the likelihood that the insulator will electrically break down due to the accumulation of electrical charge, and/or the accumulation of other materials on the surface of the insulator. In a preferred embodiment, the first node is an anode, and the second node is a cathode. Alternatively, the first node may be the cathode and the second node may be the anode.

The present disclosure will be more fully understood from the following detailed description of the examples thereof, taken together with the drawings in which:

### BRIEF DESCRIPTION OF THE DRAWINGS

FIG. 1 is a schematic, pictorial illustration of a catheter-based position-tracking and X-ray ablation system, in accordance with an example of the present disclosure;

FIG. 2 is a schematic, side view of an X-ray ablation balloon of the catheter of the system of FIG. 1, in accordance with an example of the present disclosure; and

FIG. 3 is a flow chart that schematically illustrates a method for X-ray ablation using the system of FIG. 1, in accordance with an example of the present disclosure.

### DETAILED DESCRIPTION OF EXAMPLES

### Overview

An X-ray radiotherapy catheter for ablating tissue may, in principle, be produced as, for example, a catheter that includes a miniature X-ray tube fitted at a distal end of the catheter. Such a device has benefits over, for example, brachytherapy using a radioactive element. Practically, however, the availability of such a catheter for ablation treatment is hindered, partly due to the availability of other radiotherapy methods, as well as the very high complexity of the miniature apparatus required to be fitted to the distal end of the catheter.

Examples of the present disclosure that are described hereafter provide an X-ray radiotherapy technique and a radiotherapy catheter that are relatively simple to realize. In one example, a provided X-ray radiotherapy probe for insertion into a cavity of an organ of a body of a patient comprises an expandable capsule, e.g., an expandable balloon, which is fitted at a distal end of the probe. The expandable capsule comprises a high voltage plasma discharge device, the device configured to cause emission of X-rays by electrical excitation of a noble gas that fills the expandable capsule, thus creating X-ray-emitting plasma that X-ray ablates a wall tissue of the cavity. An external high voltage generator is wired to the discharge device to apply high-voltage signals that electrically excite the plasma.

In one example, the discharge device comprises two insulated wires that extend into an interior of a capsule, or a balloon, the wires ending in a respective a pair of electrodes, each electrode being electrically fed by a respective wire. The capsule, or balloon, is filled with an inert noble gas (e.g., argon, neon, krypton, or xenon). In particular, the balloon can be expanded with the inert noble gas. In some examples, the expansion of the capsule may be assisted by additional force provided, for example, by self-expanding splines.

Applying high voltage (e.g., between 50 kV and 100 kV) between the electrodes in the environment of the noble gas inside the capsule/balloon causes arcing between the electrodes. The arcing discharges plasma that emits X-ray radiation in the process. For example, as described below, krypton plasma emits K-shell X-rays having energies in the approximate range of 13-16 keV. As further shown below, X-rays of 13-16 keV have a penetration depth into tissue that ranges between about two to four millimeters, which is an optimal depth range for clinical applications such as cardiac ablation (in addition to known applications for which such a technique is suitable, including tumor treatment).

In some examples, to improve safety, the capsule/balloon is covered with an external layer (e.g., a second balloon).

In the context of the present disclosure and in the claims, the terms “about” or “approximately” for any numerical value or range indicates a suitable dimensional tolerance that allows the part or collection of components to function for its intended purpose as described herein.

### System Description

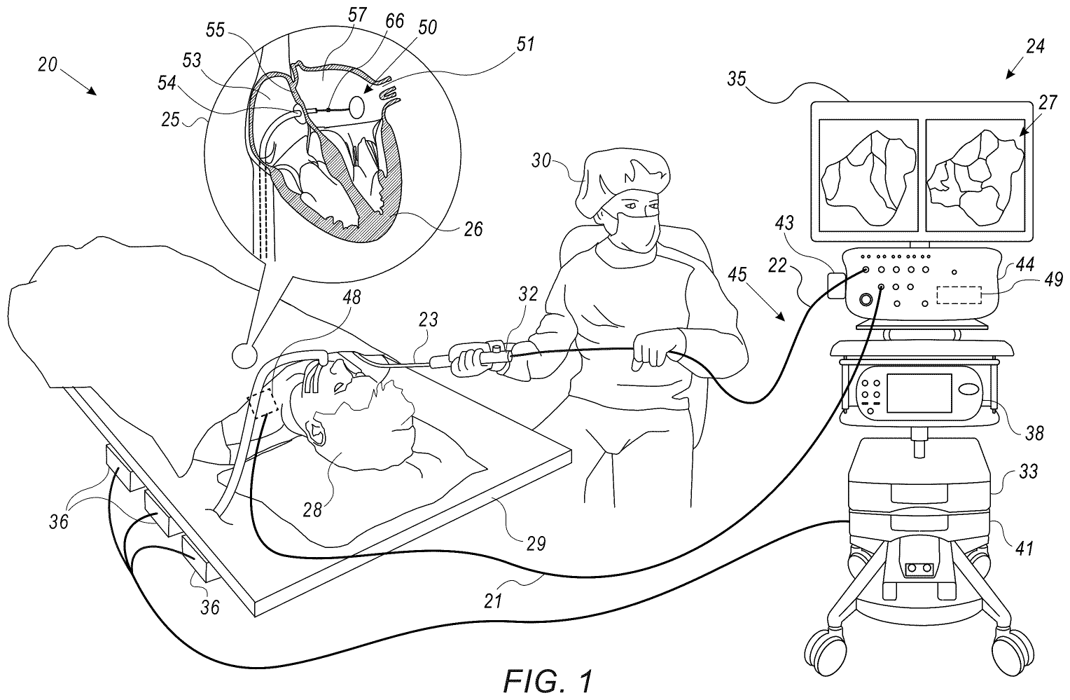

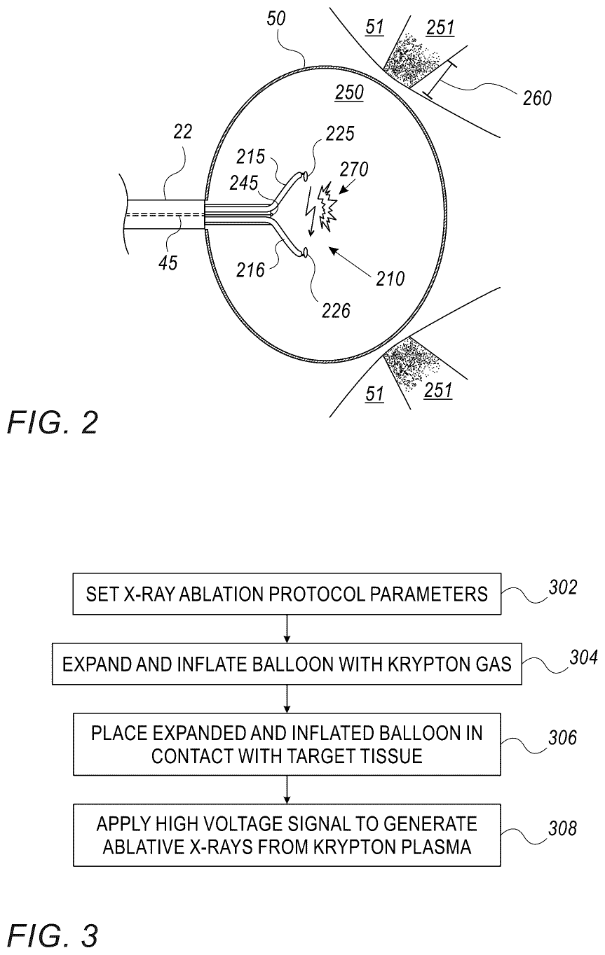

FIG. 1 is a schematic, pictorial illustration of a catheter-based position-tracking and X-ray ablation system 20, in accordance with an example of the present disclosure. In the example shown, system 20 is used for X-ray ablation of an ostium 51 of a PV (shown in inset 25) with a balloon 50 (one example of an expandable capsule 50 that can be used) located inside a left atrium 57 of a heart 26. To this end, balloon 50 is fitted with an X-ray source (e.g., a voltage discharge device), shown in FIG. 2, for the X-ray to penetrate target ostium 51 tissue locations and ablate the tissue to a given degree (e.g., depth), as described below. To perform ablation, a physician 30 advances the balloon into the left atrium and then expands (e.g., using splines) balloon 50. The balloon is expanded (e.g., the balloon self expends by Nitinol splines returning to a preformed shape as the balloon exits a sheath 23).

A noble gas with a such as krypton may be directed into balloon 50 with pressure below atmospheric, e.g., a weak vacuum. To this end, as one option among at least several possible, a pump 43 on console 24 of system 20 aspires the balloon and then pumps the noble gas via a pipe 45 running inside a catheter 22 (one example of an invasive probe 22, that can be used), which is typically at sub atmospheric pressure and is not involved in maintaining the balloon fully expanded.

The physician brings the expanded balloon with, for example, krypton into contact with ostium 51, and then the X-ray source inside the balloon (seen in FIG. 2) is excited to generate X-ray emitting krypton plasma.

As seen, balloon 50 is fitted at a distal end of catheter 22, which is configured to carry out the cardiac procedure. System 20 comprises a Console 24 comprises a high voltage generator 49 to generate, with high-voltage signals, the krypton plasma inside balloon 50. In some examples, a patient interface unit (PIU) 44 is connected to high voltage generator 49 that provides an electrical interface for the equipment included in system 10.

Console 24 further comprises a processor 33, typically a general-purpose computer, with suitable front end and interface circuits for applying high-voltage signals via catheter 22 (to create the krypton plasma) and for controlling the other components of system 20 described herein. Console 24 further comprises a user display 35, which is configured to receive graphical and/or textual display items from processor 33, such as a map 27 of heart 26, and to display map 27.

In some examples, console 24 is used with additional catheters, such as an electro-anatomical (EA) mapping catheter (not shown). To this end, console 34 comprises a recording unit 38, which is configured to record in the event of failure in the EA mapping system and/or a failure to pace in certain electrodes. Patient interface unit (PIU) 44, may be configured to produce a signal indicative of the location. PIU 44 may be configured to perform computations and/or process electrocardiogram (ECG) signals that are acquired.

In some examples, as seen in inset 25, prior to performing the X-ray ablation procedure, a physician 30 inserts one or more catheters through the vasculature system of a patient 28 lying on a table 29, so as to perform EA mapping of the tissue in question of heart 26. Based on the EA mapping, physician 30 plans the X-ray ablation.

In the present example, physician 30 intends to perform an X-ray ablation procedure at an intended location on the surface of left atrium 57 of heart 26. Optionally, the physician uses a handle 32 to (i) insert sheath 23 through the vasculature system of patient 28 into a right atrium 53 of heart 26, (ii) puncture, with the sheath, a hole 54 (also referred to herein as a transseptal hole) in a septum 55 between right atrium 53 and left atrium 57, (iii) thread sheath 23 through hole 54 into the intended location in left atrium 57, (iv) advance the folded balloon inside sheath 23 into the intended location in left atrium 57, (v) expand and inflate balloon 50 outside sheath 23, and (vi) perform the X-ray ablation procedure by placing the balloon in contact with the intended tissue and applying the high voltage signal to generate the X-rays that ablate tissue. In another example, the left atrium may be reached more directly through the aorta.

In some examples, system 20 comprises one or more patch electrodes 48, of which one is shown, which is attached to the skin of patient 28 and is electrically connected to PIU 44 via a cable 21, for example, to measure ECG signals {signals not shown).

In some examples, the position of balloon 50 in the vasculature and heart 26 of patient 28 is measured using a magnetic position sensor 66 of a magnetic position tracking system. In the present example, console 24 and/or PIU 44 comprises a driver circuit 41, which is configured to drive magnetic field generators 36 placed at known positions external to patient 28 lying on table 29, e.g., below the patient's torso. The position sensor is coupled to the distal end, and is configured to generate position signals in response to sensed external magnetic fields from field generators 36. The position signals are indicative of the position of the distal end of catheter 22 in the coordinate system of the position tracking system.

This method of position sensing is implemented in various medical applications, for example, in the CARTO™ system, produced by Biosense Webster Inc. (Irvine, California) and is described in detail in U.S. Pat. Nos. 5,391,199, 6,690,963, 6,484,118, 6,239,724, 6,618,612 and 6,332,089, in PCT Patent Publication WO 96/05768, and in U.S. Patent Application Publications 2002/0065455 A1, 2003/0120150 A1 and 2004/0068178 A1.

In some examples, processor 33 typically comprises a general-purpose computer, which is programmed in software to carry out the functions described herein. The software may be downloaded to the computer in electronic form, over a network, for example, or it may, alternatively or additionally, be provided and/or stored on non-transitory tangible media, such as magnetic, optical, or electronic memory.

This particular configuration of system 20 is shown by way of example, in order to illustrate certain problems that are addressed by examples of the present disclosure and to demonstrate the application of these examples in enhancing the performance of such a system. Examples of the present disclosure, however, are by no means limited to this specific sort of example system, and the principles described herein may similarly be applied to other sorts of medical systems. In particular, a rigid wall capsule, which is configured to be expanded when outside sheath 23, for example by being made in a modular fashion, may be used instead of a balloon.

Intracardiac Catheter with X-Ray Emitting Probe

FIG. 2 is a schematic side view of X-ray ablation balloon 50 of catheter 22 of the system 20 of FIG. 1, in accordance with an example of the present disclosure. As seen, a voltage discharge device 210 is comprised inside the balloon, in the form of two insulated wires (215, 216) that extend into an interior of the balloon, ending in a respective pair of electrodes (225, 226) that are electrically fed by the wires. The capsule or balloon is filled with an inert noble gas (250) (e.g., krypton) via pipe 45 having an outlet 245 inside the balloon.

When a high voltage (e.g., between 50 kV and 100 kV) is applied between electrodes 225 and 226 in the environment of the noble gas 250, arcing occurs between the electrodes. The arcing discharges plasma that emits X-ray radiation 270 in the process. As a result, when applied to PV isolation, X-rays can penetrate ostium 51 over an entire circumference of the ostium to ablate tissue 251 to a depth 260 into the myocardium. The ablated tissue cuts off an arrhythmogenic excitation that caused an atrial fibrillation (AFib).

As seen in FIG. 1, in some examples, the position of balloon 50 in the vasculature and heart 26 of patient 28 is measured using a magnetic position sensor of a magnetic position tracking system t that relates the balloon location to an electro-anatomical map the system built. Other ways to know when balloon 50 s in the correct location to initiate X-ray ablation include using Fluoroscopy, examining intracardiac electrograms indicative of arrhythmogenic tissue, and electrically tracking the balloon position (e.g., using one or more electrodes disposed at the distal end).

FIG. 2 is simplified for the sake of clarity of presentation of the concept of the high voltage plasma discharge device comprised in the balloon. Therefore, other elements that may be part of a balloon catheter, such as mechanical elements used for expanding and collapsing the balloon, are omitted.

Moreover, the electrode shape is conceptual, whereas actual electrode shape and geometry may vary. For example, the electrodes may be arranged in concentric geometry, with a center cathode and a ring anode, so as to ionize the noble gas in a ring-shaped geometry.

### Selection of the Noble Gas

Penetration depth is defined as the depth at which the intensity of the X-ray radiation inside the material falls to 1/e (about 37%) of its original value at (or more properly, just beneath) the surface. The X-ray penetration depth in the range 13-16 keV ranges between above 3 mm and above 5 mm. For muscle tissue, the penetration depth is slightly smaller by several percent (based on NIST attenuation tables), i.e., in the range of approximately 3-5 mm.

The krypton K-shell X-ray emission lines (He-like and H-like) occur at about energies 13-16 keV, and more precisely, as listed in Table I below:

TABLE I

| Kr lines | Energy [keV] |
|---|---|
| Kr He-α | 13.1 |
| Kr He-β | 15.4 |
| Kr Ly-α | 13.5 |
| Kr Ly-β | 15.9 |
| Kr He-γ | 16.2 |

The X-ray emission lines for xenon fall in the range of 30-40 keV, approximately, which yields a tissue penetration length range of 3-4 cm. For argon, the X-ray emission lines fall in the range of 6-8 keV, approximately, which yields a tissue penetration length range of a few to several hundred microns.

### Method of X-Ray Ablation Using a Plasma Discharge Probe

FIG. 3 is a flow chart that schematically illustrates a method for X-ray ablation using system 20 of FIG. 1, in accordance with an example of the present disclosure. The method begins at X-ray ablation protocol selection step 302, in which physician 30 sets (e.g., selects and/or adjusts) an X-ray ablation protocol according to the clinical equipment. For example, the protocol gives a time duration based on the required depth of ablated tissue entered by the physician, or based on the type of arrhythmia and/or location the ablation site selected by the physician, e.g., from a list.

At a balloon catheter expansion step 304, physician 30 advances the folded balloon inside a cardiac chamber and then expands balloon 50 (e.g., using the aforementioned self-expanding splines) and fills the balloon with krypton gas at sub-atmospheric pressure.

Next, at a balloon catheter placement step 306, physician 30 places the expanded balloon in contact with the intended tissue, such as against ostium 51.

Finally, at an X-ray ablation step 308, the physician activates the system to apply high voltage signals, according to the selected protocol, to generate the X-rays to ablate tissue.

### EXAMPLES

### Example 1

A system (20) for tissue ablation includes a probe (22), an expandable capsule (50), a voltage discharge device (210), and a generator (49). The probe is configured to be inserted into a cavity of an organ of a patient. The expandable capsule is fitted at a distal end of the probe and configured to be expanded within the cavity and to be filled with a gas (250). The voltage discharge device is fitted inside the expandable capsule and is configured to create plasma by electrical excitation of the gas that fills the expandable capsule, the plasma emitting X-rays (270), so as to ablate tissue in the cavity using the X-rays. The generator is wired to the voltage discharge device to apply electrical signals that electrically excite the plasma.

### Example 2

The system according to example 1, wherein the expandable capsule is an expandable balloon (50).

### Example 3

The system according to any of claims 1 and 2, wherein the voltage discharge device comprises a pair of electrodes (225, 226).

### Example 4

The system according to any of examples 1 through 3, wherein the electrodes (225, 226) are arranged in a linear geometry.

### Example 5

The system according to any of examples 1 through 4, wherein the electrodes are arranged in a concentric geometry.

### Example 6

The system according to any of examples 1 through 5, wherein the gas (250) is krypton, and the X-rays are generated from K-shell transition lines of the krypton plasma.

### Example 7

The system according to any of examples 1 through 5, wherein the gas (250) is xenon, and the X-rays are generated from K-shell transition lines of the xenon plasma.

### Example 8

The system according to any of examples 1 through 5, wherein the gas (250) is argon, and the X-rays are generated from K-shell transition lines of the argon plasma.

Although the examples described herein address cardiac ablation, the methods and systems described herein can also be used in other applications, such as renal denervation.

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A system for tissue ablation, the system comprising:
a probe, configured to be inserted into a cavity of an organ of a patient;
an expandable capsule fitted at a distal end of the probe and configured to be expanded within the cavity and to be filled with a gas;
a voltage discharge device, which is fitted inside the expandable capsule and is configured to create plasma by electrical excitation of the gas that fills the expandable capsule, the plasma emitting X-rays, so as to ablate tissue in the cavity using the X-rays; and
a generator, which is wired to the voltage discharge device to apply electrical signals that electrically excite the plasma.

2. The system according to claim 1, wherein the expandable capsule is an expandable balloon.

3. The system according to claim 1, wherein the voltage discharge device comprises a pair of electrodes.

4. The system according to claim 3, wherein the electrodes are arranged in a linear geometry.

5. The system according to claim 3, wherein the electrodes are arranged in a concentric geometry.

6. The system according to any of claims 1-5, wherein the gas is krypton, and the X-rays are generated from K-shell transition lines of the krypton plasma.

7. The system according to any of claims 1-5, wherein the gas is xenon, and the X-rays are generated from K-shell transition lines of the xenon plasma.

8. The system according to any of claims 1-5, wherein the gas is argon, and the X-rays are generated from K-shell transition lines of the argon plasma.

9. The system according to any of claims 1-5, wherein the gas inside the expandable capsule is filled to a sub-atmospheric pressure.

10. A method for tissue ablation, the method comprising:
inserting a probe into a cavity of an organ of a patient, the probe comprising an expandable capsule fitted at a distal end thereof;
filling the expandable capsule with a gas; and
using a voltage discharge device fitted inside the expandable capsule, creating plasma by electrical excitation of the gas that fills the expandable capsule, the plasma emitting X-rays, so as to ablate tissue in the cavity using the X-rays.

11. The method according to claim 10, wherein the expandable capsule is an expandable balloon.

12. The method according to claim 10, wherein the voltage discharge device comprises a pair of electrodes.

13. The method according to claim 12, wherein the electrodes are arranged in a linear geometry.

14. The method according to claim 12, wherein the electrodes are arranged in a concentric geometry.

15. The method according to any of claims 10-14, wherein the gas is krypton, and the X-rays are generated from K-shell transition lines of the krypton plasma.

16. The method according to any of claims 10-14, wherein the gas is xenon, and the X-rays are generated from K-shell transition lines of the xenon plasma.

17. The method according to any of claims 10-14, wherein the gas is argon, and the X-rays are generated from K-shell transition lines of the argon plasma.

18. The method according to any of claims 10-14, wherein the gas inside the expandable capsule is filled to a sub-atmospheric pressure.
